(19) United States
(12) Patent Application Publication
(10) Pub. No.: US 2004/0045047 A1
(43) Pub. Date: Mar. 4, 2004

(54) Method for plant gene transfering by electrical shock and ovary injection

(76) Inventor: Pan-Chi Liou, Taichung (TW)

Correspondence Address: Hart, Baxley, Daniels & Holton, Fifth Floor, 59 John Street, New York, NY, 10038, US

(21) Appl. No.: 10/228,365

(22) Filed: Aug. 27, 2002

(51) Int. Cl. C12N 15/82; C12N 15/87

(52) U.S. Cl. 800/278; 435/468

(57) ABSTRACT

A method for plant gene transfer by ovary injection and electrical shock. First the stigma is cut off after the plant is pollinated. A tiny DNA passage is then defined by penetrating an injection needle from the cut-off at the style and toward the locule inside the ovary. An exotic DNA containing gene(s) is injected into the locule via the passage. An electric field is set up with the center of the ovary being negative and the periphery positive. The DNA which is negatively charged in nature will move outward in such an electric field. This arrangement increases possibility of DNA recombination.

### FIELD OF THE INVENTION

The present invention relates to a technique about gene transferring, and more particularly, to a method for plant gene transferring by electrical shock and ovary injection.

### BACKGROUND OF THE INVENTION

A conventional way of plant gene transferring includes the following steps: (1) cloning the gene or genes from proper sources and recombining the cloned gene(s) with proper DNA vector (plasmid) used as a gene carrier; (2) transferring DNA carrier harboring the cloned gene(s) into plant cells by proper methods so as to form transgenic cell, including particle gun bombardment method, Agrobacterium-mediated method, microinjection, electroporation, virus-mediated method, and PEG method (polyethylene glycol method) etc.; (3) the above mentioned methods all require the aid of plant tissue culture to transfer the transgenic cells by way of regeneration into transgenic plants having roots, stems, and leaves; (4) examining and screening for the successful and good transgenic plants. The above mentioned methods all have their own characteristics, however, they also have some common shortcomings which are not ready for being overcome. One of the shortcomings is that the efficiency of transferring is not always satisfied. Usually a transgenic process is more likely to be successful, if a tissue culture technique with high regeneration efficiency is available. Unfortunately, in many of the cases the tissue culture technique has not yet been well established so far. This is one of the main reasons making the success of gene transfer so rare in many crops. From this point of view, a new approach of transgenic process being able to enhance transferring efficiency and not relying on the aid of tissue culture will be very valuable and useful.

### SUMMARY OF THE INVENTION

The object of the present invention is achieved by (1) at a suitable interval of time after pollination, injecting the DNA containing exotic gene(s) into the locule of plant ovary according to a series of operation steps to let DNA surround the ovule(s)inside the locule; (2) by using a controlling device to proceed an electrical shock to the ovary under the controlled voltage, current and timing, in order to form a temporary electric field in the ovary with negative electrode in the center and positive electrode distributing on outside arounding the ovary walll. The injected DNA which is negatively charged in nature will move outward from the central locule toward the directions of ovary wall so as to increase the opportunity to enter into the ovule, and further into the egg cell. This makes DNA recombination between exotic DNA and the fertilized egg's chromosome (i.e. gene tranfer) possible. The efficiency of gene transfer can be increased.

The present invention will become more obvious from the following description when connected with the accompanying drawings which show, for purposes of illustration only, a preferred embodiment in accordance with the present invention.

### BRIEF DESCRIPTION OF THE DRAWINGS

FIG. 1 is a flow chart of the steps of the method of the present invention;

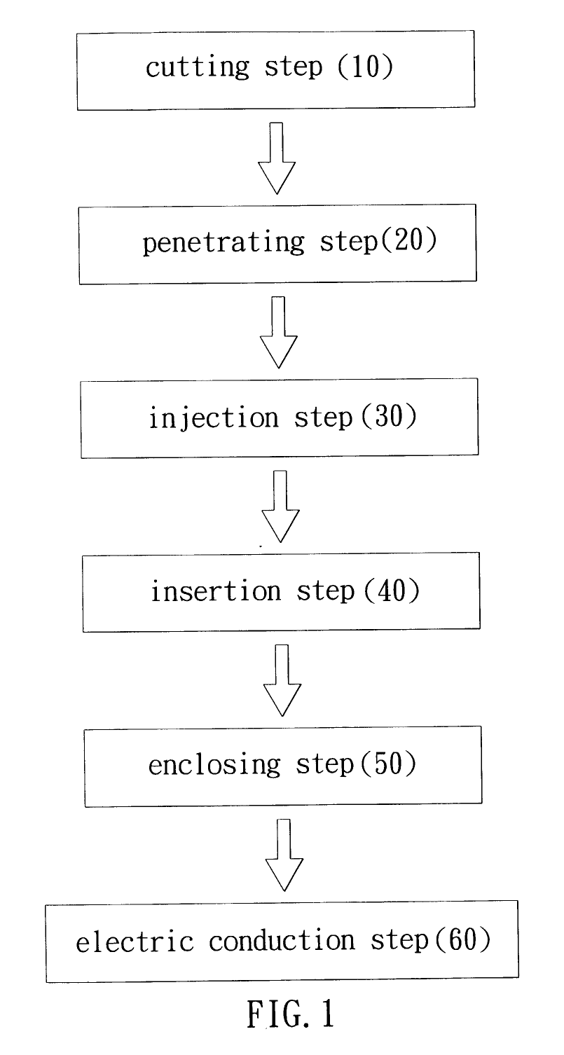

FIG. 2 shows parts of a flower of a plant, and

FIG. 3 shows the steps of cutting, penetrating, and injection;

FIG. 4 shows another way of the step of the penetrating; and

FIG. 5 shows the step of insertion, enclosing and electrical conduction of the present invention.

### DETAILED DESCRIPTION OF THE PREFERRED EMBODIMENT

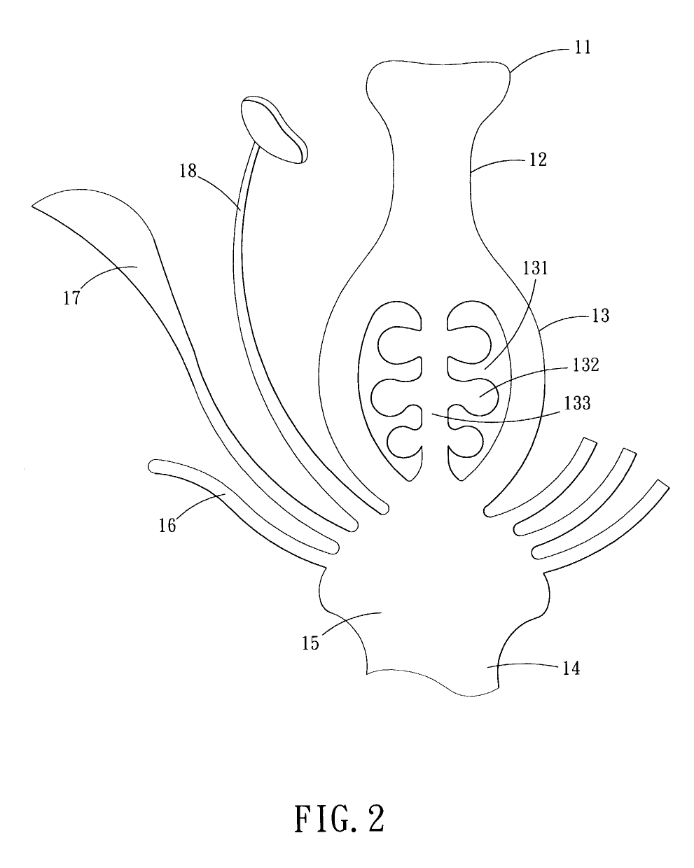

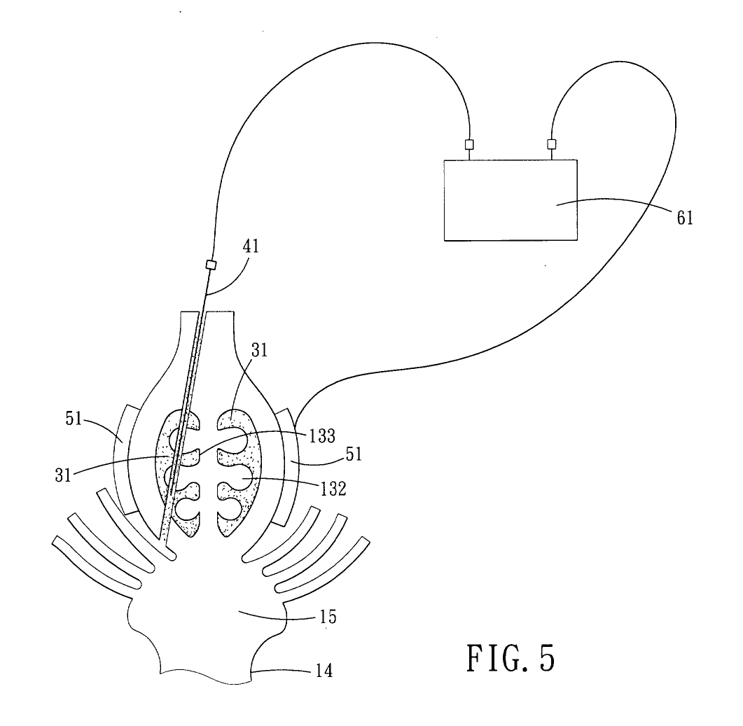

Referring to FIGS. 2 to 5, the method for transferring gene of plant of the present invention comprises six steps which are cutting step 10: cutting the stigma off at a suitable interval of time after pollination; penetrating step 20: making a tiny passage or tunnel by using an injection needle to penetrate from style toward locule; injection step 30: injecting the DNA from outside into the locule of ovary via the passage; insertion step 40: inserting electrically conductive piece into the passage to be used as a negative electrode ; enclosing step 50: enclosing the wall of the ovary with electrically conductive layer so as to form a positive electrode; electrical conduction step 60: connecting the positive and negative electrodes to an electric device and proceeding an electrical shock to create a special electric field. The six steps are operated continuously.

For the cutting step 10, referring to FIG. 2 which shows the flower of plant and includes stigma 11, style 12, ovary 13, locule 131, ovule 132, placenta 133, floral stalk 14, receptacle 15, calyx 16, petal 17, stamens 18. After being pollinated, when the pollen tubes reach the ovule(s), using a certain tool to cut off the stigma 11 at the position of the style 12 as shown.

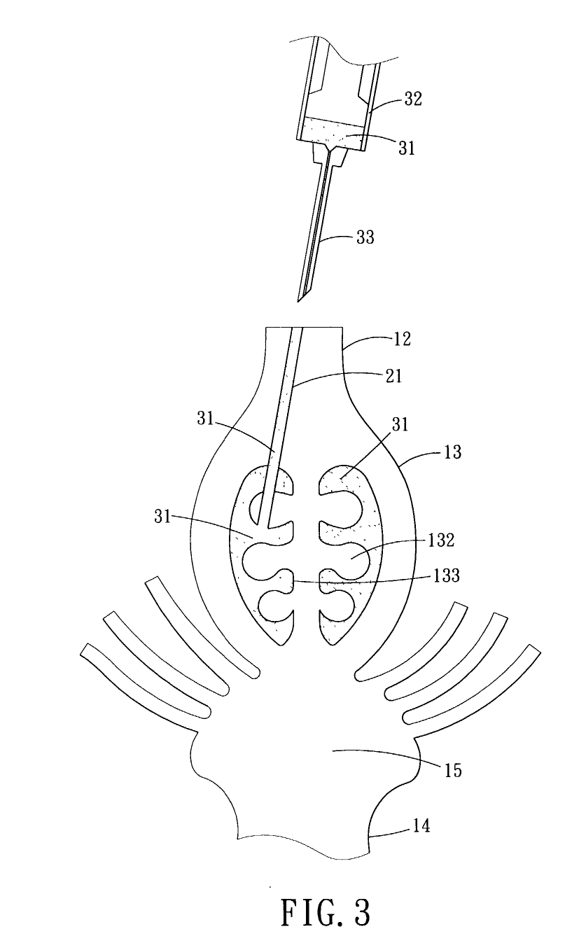

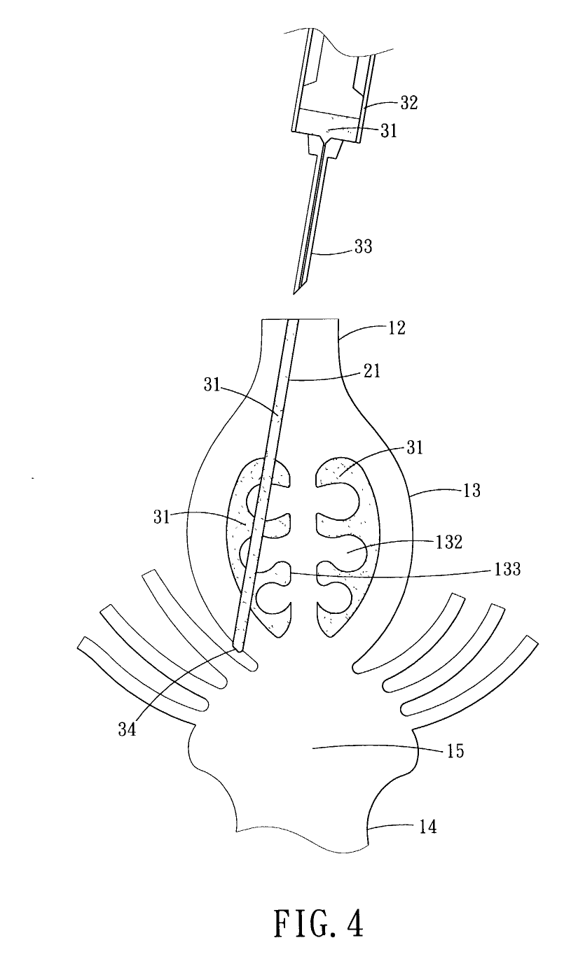

For the penetrating step 20, a tiny passage 21 is defined between the style 12 and the locule 131 by using an injection needle to penetrate into the cutting place of the style 12 and toward the ovary 13 till the locule as shown in FIG. 3, or going further through the locule 131 to penetrate the ovary wall and make an opning 34on it as shown in FIG. 4, and then pulling the needle out to define the tiny passage 21.

For the injection step 30, the DNA 31 is injected into the locule 131 by a needle 33 of the injection device 32 via the passage 21 to allow the DNA 31 to surround the ovule 132. If the passage 21 is defined by penetration as shown in FIG. 4, some DNA 31 could flow out from the opening 34, but this does not interrupt the result of gene transfer according to repeated tests.

For the insertion step 40, inserting an electrically conductive piece 51 such as a Platinum wire, into the passage 13 to form the negative electrode.

For the enclosing step 50, an electrically conductive layer 51 is enclosed the ovary 13 which is to make a circular positive electrode.

For the electrical conduction step 60, connecting the positive and negative electrodes as defined in steps 40 and 50 to a special electronic controlling device 61 which will give an electrical shock to form a special electrical field determined by the combination of certain voltage, current and timing.

DNA 31 is negatively charged by its chemical nature and will move outward positive electrode in the electric field. Roughly said, in this situation the DNA molecules inside locule will move from the center to the surroundings. The opportunity for DNA to enter the egg cell inside ovule 132 is increased, especially when the egg-sperm fusion (i.e. fertilization) occurs if the timing is well managed. Around the time of fertilization, some of the egg's cell wall is thin and the egg cell is somewhat similar to a protoplast. This characteristics makes the DNA 31 easy to penetrate through the cell wall, especially at the moment when ferlization occurs. As a result, the possibility that exotic DNA is combined into the chromosome of the fertilized egg, i.e. gene transfer, can be increased. The transgenic fertilized egg will then become a transgenic seed through nature development, and a transgenic plant can be produced by the transgenic seed through germination and vegetative growth. The whole procedures do not need the aid of tissue culture.

Obviously, in this method the occurrence of gene transfer depends on 5 main factors including: 1. the existence of injected DNA 31 which contains exotic gene(s); 2. sperm cells traveling toward the egg inside the extending pollen tubes after pollination; 3. egg cell(s) inside ovule(s) 132; 4. the occurrence of egg-sperm fusion (i.e. fertilization) inside ovule; and 5. electric shock treatment which increases the movement of the exotic DNA 31 inside the locule. To manage all these factors in order to make gene transfer being able to occur with high possibility, an accurate timing is very important for all the steps in the method of the present invention.

The present invention not only achieve the purpose of gene transfer, but also includes the following five advantages:

1. Increase the rate of success of plant gene transfer: the DNA is injected into the ovary at suitable time after pollination and then treated by electric shock to dramatically increase the rate of success of gene transfer.

2. Reduce the cost of the research of genetic engineering: the present invention adopts the natural way of plant breeding process to achieve the purpose of gene transfer. It is simple and easy to do. Besides, it is found that using this method the efficiency is higher than the conventional ways used to transfer gene(s) into plants. This method does not need the aid of tissue culture to produce a complete transgenic plant from a transgenic cell. Such a characteristics is quite valuable and helpful for time and expenses saving, especially for those plant species in which the technique of tissue culture has not yet been well established.

3. Benefit the environmental safety: The method of the present invention neither needs tissue culture technique nor rely on baterium mediation in all the procedures, therefore the problem of environmental contamination and pollution can be greatly reduced. Therte is also no damage to the eco-system, even the working procedures of this invention are all performed in the open field.

4. Easy to practice and perform: Using the method of the present invention, the practice and skill of gene transfer is easy to learn and teach. Once the technique for a specific plant has been well determined and established, including the timing of DNA injection and the condition of electrical shock, the experiment of gene transfer could become a routine work and be easily performed by an assistant worker who is even without the background of genetics and molecular biology. The main part of experiment work can be done in the field or green house using a syringe of suitable size (or an injection device originally designed for gas chromatography analysis) and an electrical shock inducing device. No expensive instrument and equipment is needed.

5. Particularly suitable for those plants having multiple seeds produced in one fruit, if the fruit is originated from one single pollinated flower such as tomato and watermelon: Since ovary is the basic unit to deal with in this method, this method is especially useful and valuable for those plants with multiple seeds produced in one single-flower-originated-fruit. Appraently the rate of success can be enhanced, if multiple seeds can be obtained from one single experimental treatment. The more the seeds can be obtained from one treated ovary (flower), the higher the rate of success can be expected. In agriculture, there are many crops bear multiple-seeds-containing fruits which are developed from one single pollinated flower. Their species distribute in different taxonomic families, including Orchidaceae, Cucurbitaceae, Leguminosae, Solanaceae, Rosaceae, Cruciferae, Rutaceae, Myrtaceae, Liliaceae, Passifloracae, Oxalidaceae, Vitaceae, Actinidiaceae, and Caricaceae etc. We estimate that ca. ⅔ of horticultural crops are of this type, and most of these crops still lack good tissue culture research. In fact tissue culture research even has not yet been initiated in many of these crops, and so does the genetic engineering work, therefore, the method of this invention is going to have a huge potential for application in many crops'genetic improvement.

While we have shown and described the embodiment in accordance with the present invention, it should be clear to those skilled in the art that further embodiments may be made without departing from the scope of the present invention.

What is claimed is:

1. A method for plant gene transfer by electrical shock and ovary injection, wherein the stigma is cut off after the flower is pollinated and pollen tubes have properly developed, a passage is defined between the style and ovary, exotic DNA is then injected via the passage into ovary to fill in the locule. An electrically conductive piece inserted via the passage deep into the locule is to be used as negative electrode, and an electrically conductive layer tightly enclosing the ovary wall as positive electrode. An electric field is then produced by an electrical device with specific voltage, current, and length of time. The electric shock makes the exotic DNA moves outward, i.e. from the central locule toward the wall of ovary, so as to increase the possibility for DNA to enter the egg inside the ovule. As a result, the possibility that exotic DNA is combined into the chromosome of the fertilized egg, i.e. gene transfer, can be increased. The transgenic fertilized egg will then become a transgenic seed through nature development, and a transgenic plant can be produced by the transgenic seed through germination and vegetative growth.

2. The method as claimed in claim 1, wherein the electrically conductive wire can be a Platinum wire and is used as the negative electrode, the electrically conductive layer enclosing the ovary is used as he positive electrode.

3. The method as claimed in claim 2 [symbol] wherein an electronic controlling device is connected to the positive electrode and the negative electrode, an electric field with negative electrode near the center of the ovary and positive electrode at the periphery of the ovary is then created by specific voltage, current and length of time, such an electric field makes the DNA which is negatively charged in nature moving outward so as to increase the opportunity of gene transfer during the process of fertilization.

4. The method as claimed in claim 1, wherein the passage is defined by penetrating an injection needle from the position of the cut-off on the style toward the locule inside the ovary, and the needle is then pulled out.

5. The method as claimed in claim 1, wherein the passage is defined by penetrating an injection needle from the position of cut-off on the style toward the locule and further penetrating through the wall of ovary, and the needle is then pulled out.
